(19) United States
(12) Patent Application Publication
(10) Pub. No.: US 2007/0060828 A1
(43) Pub. Date: Mar. 15, 2007

(54) Ecg system and method for the large-surface measurement of ecg signals

(75) Inventors: Markus Zabel, Berlin (DE); Hans Koch, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: Charite-Universitaets-Medizin, Berlin (DE)

(21) Appl. No.: 10/567,411

(22) PCT Filed: Aug. 6, 2004
(86) PCT No.: PCT/DE04/01794

§ 371 Date: Feb. 6, 2006

(30) Foreign Application Priority Data
Aug. 7, 2003 (DE) 10336809.4

(51) Int. Cl. A61B 5/04

(52) U.S. Cl. 600/509

(57) ABSTRACT

The invention relates to an ECG system for the large-surface measurement of ECG signals which is characterized by a first measuring device (10) for generating a first set of readings comprising at least one derivation of the electrical impulses of the heart. At least one site of derivation of the first measuring device (10) during recording of the large-surface ECG signals is variable. The system further comprises a second measuring device (20) for generating a second set of readings comprising at least one derivation of the electrical impulses of the heart. The site of derivation of the second measuring device (20) during recording of the large-surface ECG signals is spatially invariable in order to obtain continuous readings. A data processing system (30) is provided with means for synchronizing at least two signals of the first set of readings that are detected at different times with at least one continuously detected signal of the second set of readings. The inventive system allows for the large-surface detection of ECG signals and for their efficient use in everyday hospital routine.

The invention relates to an ECG system having the features of claim 1 and to a method for large-surface measurement of ECG signals according to claim 14.

The 12-channel ECG is the accepted standard in everyday hospital routine. In particular, the electrode positions are accurately fixed on the body. Again, the way in which the ECG signals are read off, computed and graphically displayed is stipulated. A detailed presentation is known from “Comprehensive Electrocardiography—Theory and Practice in Health and Disease”, Volume 1, publishers P. W. Macfarlane and T. D. Veitch Lawrie, Pergamon Press, New York, 1989, in particular Chapter 11 “Lead Systems”.

The electric potential is measured at six thoracic wall positions (V₁ to V₆) when using the classical 12-channel ECG method. Added to these are six extremity leads (I, II, III, aVL, aVR, aVF). It has already been recognized here as disadvantageous that the changes in electric potential generated by the cardiac activity are spread over a large surface of the body. With specific clinical pictures there are characteristic variations in thoracic areas that are not detected by the classic ECG electrodes.

It is therefore desirable from the clinical point of view to read off ECG signals over a relatively large thorax area.

This is achieved, for example, by the so-called body surface potential mapping (BSPM), it having been demonstrated that additional clinically relevant data can thereby be determined (N. C. Flower, L. G. Horan in “Body Surface Potential Mapping”, Chapter 82 in “Cardiac Electrophysiology—From Cell to Bedside”, 3rd edition, publishers D. P. Zipes and J. Jalife, W.B. Saunders, Philadelphia, 2000).

This method, in which the electric potential is measured simultaneously at 20 to 200 electrode positions, has not so far been able to establish itself in everyday hospital routine because of its complexity and the high costs associated therewith. It is known to supplement the classic 12-channel ECG measurements with additional measurements at other electrode positions in order to improve the clinical diagnosis (for example A. P. Michaelides et al. “Improved detection of coronary artery disease by exercise electrocardiography with the use of right precordial leads” N. Eng. J. Med. 340 (1999) 5).

However, with these examinations there is no method in use that enables a sufficiently accurate synchronization of the individual signals in order to achieve a mapping in the sense of the BSPM.

Furthermore, the article by Bruno Taccardi, “Distribution of Heart Potentials on the Thoracic Surface of Normal Human Subjects”, (Circulation Research, Volume XII, April 1963) discloses a method in which diverse thorax leads are used in conjunction with a reference ECG, likewise read off at the thorax, for the purpose of ascertaining the cardiac potential at the thoracic wall of a healthy proband. However, it is necessary here to generate a calibration signal after each cardiac contraction by means of a square-wave generator, and this has the disadvantage of requiring a corresponding outlay on apparatus.

It is therefore the object of the present invention to develop a method for large-surface recording of ECG signals that can, however, be applied easily and efficiently in everyday hospital routine.

The inventive ECG system uses a first measuring means for generating a first measured data record including at least one reading of the cardiac currents, at least one lead site of the first measuring means (10) being variable during the recording of the large-surface ECG signals. Furthermore, use is made simultaneously of a second measuring means for generating a second measured data record including at least one reading of the cardiac currents, the lead site of the second measuring means being spatially invariable during the recording of the large-surface ECG signals in order to obtain continuous measurement results. Finally, the inventive ECG system has a data processing system having a means for synchronizing at least two signals, determined in a temporally offset fashion, of the first measured data record with at least one continuously detected signal of the second measured data record. It is thereby possible to synchronize at least one discontinuously obtained signal of the first measured data record by means of at least one continuously obtained signal of the second measured data record. Such a system can be used, for example, in intensive medicine. It is a simple matter to offset the measuring points spatially on a patient who is lying down.

It is advantageous here when the first measured data record includes measurements of cardiac currents that have been obtained at thorax leads (V₁-V₆). It is particularly advantageous when the first measured data record includes measurements of the cardiac currents from a temporal sequence of thorax leads (V₁-V₆) at different thorax positions. It is then possible thereby to obtain ECG data over a large surface.

One advantageous possibility for obtaining continuous measured data is when the second measured data record includes at least one measurement of the cardiac currents of an extremity lead (I, II, III, aVR, aVL, aVF). It is particularly advantageous when the second measured data record includes signals of the cardiac currents of all the extremity leads (I, II, III, aVR, aVL, aVF).

In an advantageous refinement of the inventive ECG system, the synchronization is performed with the aid of at least one prominent signal pattern of the second measured data record.

It is advantageous here when the means for synchronizing uses the signal of a R wave in the second measured data record for the purpose of synchronization. It is particularly advantageous when the means for synchronizing uses the signal of the rise in the R wave in the second measured data record for the purpose of synchronization.

It is advantageous, furthermore, when the means for synchronizing uses prominent signal markers of a number of measured ECG channels.

A further advantageous refinement of the inventive ECG system has a filter, a means for averaging and/or for determining the median for signals of the first measured data record and/or of the second measured data record. It is thereby possible to determine characteristic heartbeats that are used for the synchronization.

It is also advantageous when the ECG system has a means for correcting the baseline of individual cardiac currents.

An embodiment of the inventive ECG system advantageously has a data processing system that uses the amplitude values of all the thorax readings to determine a graphic display of the instantaneous potential distribution automatically for any desired instant of a measurement relative to a time reference obtained by means of a signal of the second measured data record.

It is advantageous here when the graphic display is a QRST integral map display.

It is advantageous for recording the large-surface ECG signals when the first measuring means and/or the second measuring means are/is arranged in a contrivance, in particular a vest, that can be worn on the human body. A long-term ECG examination, for example, is thereby possible.

It is advantageous for checking the effectiveness of a measurement when a variance of measurement results can be ascertained as a validity characteristic by means of the data processing system. It is particularly advantageous here when the variance of the measurement results can be ascertained with the aid of a measure of specific ECG potential levels, in particular R-R intervals, QT times and/or of a comparison of a mean value of a measure of an ECG potential level of one measurement phase with the mean value for measures of ECG potential levels of all the measurement phases.

The object is also achieved by means of a method for large-surface recording of ECG signals having the features of claim 17.

Two data records that can be related to one another efficiently are generated by recording at least one first measurement of the cardiac currents with the aid of a first measuring means, at least one lead site of a first measuring means being varied during recording of the large-surface ECG signals, and by simultaneously recording at least one second measurement of the cardiac currents with the aid of a second measuring means, the lead site of the second measuring means being spatially invariable during recording of the large-surface ECG signals for the purpose of continuous measurement. Immediately or at a later instant, at least two signals, determined in a temporally offset fashion, of the cardiac currents of the first measured data record are automatically then synchronized in a data processing system with at least one continuously determined signal of the second measured data record of the cardiac currents. It is advantageous here when at least two first readings are obtained on the thorax in a fashion separated by an intercostal spacing, in particular for the purpose of simulating a body surface potential mapping.

The inventive system, with the aid of which it is possible to produce approximations of body surface potential mappings (BSPM) by using generally available digital 12-channel ECG systems, is described below with the aid of exemplary embodiments. These pseudo BSPMs include the majority of the average spatial temporal information of a single characteristic heartbeat. The fundamental signal processing is described in detail. The algorithms can be added in a simple way to the software of commercial 12-channel ECG units.

Body surface potential mapping (BSPM) is demonstrably a clinically relevant method that raises the diagnostic performance by comparison with the standard 12-channel ECG. An overview is given by (see N. C. Flowers, L. G. Horan: “Body Surface Potential Mapping” in D. P. Zipes, J. Jalife (eds): “Cardiac Electrophysiology: From Cell to Bedside”, 2nd ed., Philadelphia, WB Saunders, 1995, pp. 1049-1067; N. C. Flower, L. G. Horan in “Body Surface Potential Mapping”, Chapter 82 in “Cardiac Electrophysiology—From Cell to Bedside”, 3rd edition, publisher D. P. Zipes and J. Jalife, W.B. Saunders, Philadelphia, 2000; “Comprehensive Electrocardiology—Theory and Practice in Health and Disease”, Vol. 1; “Comprehensive Electrocardiography—Theory and Practice in Health and Disease”, Volume 1, publishers P. W. Macfarlane and T. D. Veitch Lawrie, Pergamon Press, New York, 1989, in particular Chapter 11 “Lead Systems”) and the source data contained therein.

The reason for such an improved detection and separation of pathophysiological cardiac functions by means of BSPM is grounded in the substantially larger number of measuring positions of the electrodes that are fastened on the thorax. FIGS. 1a and 1b provide an immediate impression of the improved coverage of the thorax and the consequently improved detection of important spatial components of the distribution of electrical potential by means of BSPM (FIG. 1b), which would otherwise have escaped the six standard breast electrodes of the 12-channel ECG (FIG. 1a). At different instants, the potential distribution is frequently varied substantially in spatial terms. Consequently, spatially and temporally important properties cannot be acquired by the 12-channel ECG, but this is possible with the aid of the BSPM.

It should be mentioned at this juncture that FIG. 1b does not illustrate the usual distribution of the BSPM electrodes, but the configuration that was selected for the method presented here. For conventional BSPM, the number and distribution of the electrodes on the thorax is not standardized exactly as in the case of the six breast electrode positions of the 12-channel ECG. Although only a few concepts of the electrode configuration have been established, none of them is actually accepted throughout the world. An argument could be made to the effect that exact positioning of the electrodes is not so important for the BSPM as it is for the V₁-V₆ electrodes of the 12-channel ECG, since all the relevant properties are acquired in some way because of the mapping—if not by a specific electrode, then by one of the neighboring electrodes.

There is a compromise between the complexity (and thus the costs) and the detection of relevant information with reference to the optimum number of electrodes for BSPM. Complexity and costs are the main obstacle that has prevented BSPM from breaking through in hospital routine. However, that is the motivation of this invention: to provide a system and a method that achieve comparable—although not identical—results as does BSPM, but only with the need for a standard 12-channel ECG instrumentation.

The pseudo-BSPM method is named as such to demarcate it from true BSPM. The main difference from true BSPM is that not all the channels are read out simultaneously, that is to say the mapping is reconstructed from sequentially obtained ECG signals. On the other hand, most of the properties denoted as clinically relevant in the BSPM literature originate from averaged data and include no information on variability from heartbeat to heartbeat. Consequently, the difference between the graphic displays that are obtained by true BSPM and by the pseudo BSPM presented here are not substantial.

The aim of the present invention is to describe with the aid of an exemplary embodiment how the sequentially recorded ECG signals of a commercial 12-channel ECG system are synchronized in such a way that it is possible to compile valid approximations of BSPMs.

FIG. 2a describes customary forms of leads of a 12-channel ECG. The thorax leads (V₁ to V₆) are illustrated in the upper part of FIG. 2. The bipolar extremity leads (I, II, III) and the unipolar extremity leads (aVR, aVL, aVF) are illustrated in the lower part.

The system according to the invention has a first measuring means 10 (illustrated schematically here) that measures the signals of the thorax leads. However, at least one spatial position of the thorax leads is varied successively in the course of a complete measurement. Consequently, at least one reading V₁ to V₆ is determined at different points within a complete measurements. It is to be assumed below that all the thorax leads are spatially displaced on the thorax at the end of a first measurement section. This can be an intercostal spacing in each case, for example.

By contrast, the extremity readings, which are recorded by a second measuring means 20 (illustrated schematically) remain spatially invariable during a complete measurement. As will be further set forth in detail later, these readings serve for generating a pseudo synchronization of the thorax readings tapped spatially at different sites.

The extremity readings are also used in one embodiment of the invention for the purpose of determining at least one validity characteristic. The validity characteristic is a measure of whether the basic precondition is fulfilled for the inventive method during the entire measurement period, specifically the extensive constancy of the heartbeat pattern. This will be explained in more detail below.

The pseudo synchronization is carried out by a data processing system 30 that has, implemented as hardware or software, a means with the aid of which the measuring signals of the first measuring means 10 and of the second measuring means 20 can be synchronized with one another automatically.

The following description is focused only on a process that yields a pseudo BSPM from the use of a commercial digital 12-channel ECG system.

Data Production

The data production for the pseudo BSPM can be carried out with any standard 12-channel ECG unit if a digital data output permits a reconstitution of digitized signals for a numerical offline reconstruction.

A typical recording session includes the following different phases:

Phase 1:

Firstly, a standard 12-channel ECG recording procedure is begun, that is to say electrodes are fitted at the standardized positions of the thorax (V₁-V₆) (compare FIGS. 1a, 2) and of the extremities (I, II, III, aVR, aVL, aVF), and the 12-channel ECG signals are recorded with the aid of the measuring means 10, 20.

Phase 2:

The breast electrodes (V₁-V₆) are now displaced upward by a rib spacing, while the electrode positions of the extremities remain invariable. The 12-channel ECG signals of phase 2 are then recorded with the aid of the measuring means 10, 20. In general, the signal recordings should follow one another quickly, there being a need to ensure prevention of instances of dysrhythmia or variations in the basal heart rate.

Phases 3 to 8:

These phases are carried out in the same way, with the result being a sequential coverage of the thorax electrode positions as illustrated in FIG. 1b.

Finally, the ECG signals stored in the data processing system 30 should have a recording format such as is shown schematically for the first three phases of FIG. 3. It should be ensured that the recording in phase 1 begins with a completely standardized 12-channel ECG. As a result, no information is lost by comparison with the standard ECG; all that follows next is additional (or redundant) information.

In phase 2, the ECG traces, which are normally reserved for V₁-V₆, now contain the recorded signals of the positions 7 to 12, while the traces I, II, III, aVR, aVL, aVF contain continuous recordings of the second measuring means 20 of the extremity electrodes, whose position is unchanged.

Pseudo Synchronization

The aim of BSPM is to obtain a graphic display of the spatial distribution of the electric potential of the thorax surface for a specific instant—for example the peak QRS complex in channel V₁—by means of the data processing system 30. The normal procedure with a standard BSPM system functions such that the instantaneous signal amplitude referred to this instant is collected from the ECG signals of all the channels and generally accessible algorithms are used to put together the surface adaptation with grid points referred to the electrode positions. This surface adaptation function is then displayed either as a contour plot, a gray scale plot or a false color plot. Since all the BSPM channels operate in a truly simultaneous fashion, it is automatically ensured that all the data points that yield the graphic display have been measured at the same instant.

The situation is entirely different with the pseudo BSPM method as presented here, since the data points in the graphic displays to be constructed originate from very different instants. As shown in FIG. 3, in order to put a graphic display together it will be necessary to collect data points for the electrode positions 1 to 6, measured at the instant t₁, the positions 7 to 12, measured at the instant t₂, the positions 13 to 18, measured at the instant t₃, etc.

Thus, it is necessary to construct a single graphic display of at least eight different ECG heartbeats that have been measured with a time difference of a few minutes. Of course, such a graphic display is in no way representative of a specific instant. Nevertheless, it is possible to construct a graphic display that constitutes a good approximation of a true BSPM when the individual ECG heartbeat signals of all eight phases are “laid one over another” in a suitable way in order to form a “characteristic heartbeat”. If it is decided that properties of the ECG heartbeat pattern are maintained—even for slightly varying pulse rates—such a construction can then still include clinically relevant information. What is lost is the variability from heartbeat to heartbeat.

It should be stressed here that it is essential to fix the reference instants t₁ to t₈ (compare FIG. 3) as precisely as possible. In order to obtain the best possible approximation to true BSPM, finding the “correct” reference times must be regarded as important. This problem is to be explained in more detail by means of the following example:

FIG. 4a shows a collection of simultaneously recorded ECG signals of the QRS of selected electrode positions.

Clearly, the extremes of the individual signals do not occur during the same time. FIG. 4b shows the corresponding BSPM for the instant illustrated in FIG. 4a as a broken line.

Normally, no recording of the extremity electrodes is carried out for a BSPM. If, as in the case of the inventive system, only the 48 breast electrodes were to be recorded in a sequential way as described above, it would be difficult to decide how the individual ECG heartbeat patterns should be assigned temporally to one another, in order to lay them “correctly” over one another in the course of time, that is to say in order to obtain a similar picture as in FIGS. 4a and 4b. The realistic time information is lost because of the sequential recording. Clearly, an adjustment of the peak properties would lead to a false result, as shown in FIG. 4c and the graphic display in FIG. 4d associated therewith.

Proceeding from FIG. 4a, it is suggested to select the start of the Q wave as conforming instant. This would actually function in this example. Nevertheless, it is known that this start can vary substantially from channel to channel in many other cases, and is frequently not particularly well pronounced. If a channel/electrode were to be kept in a position, let us say V₁, during the entire sequential recording, and use were to be made, for example, of the R peak as trigger in order to synchronize all the other heartbeat signals, this would not function reliably in many cases. Furthermore, the beginning of the QRS complex of a channel is frequently not pronounced enough and produces ambiguous results.

A more reliable method that leads to better results is, in contrast, described as follows: the reliability for obtaining adequate reference instants is increased when the time marker of a number of channels is derived. Very pronounced properties can be achieved when the sum of the squares of the rate of variation of the signal amplitude a(t) over all the extremity electrodes is calculated for each time step t₁:

$$r ( {t}_{i} ) = {\sum }_{n = 1}^{6} {[ {a}_{n} ( {t}_{i} - t ) - {a}_{n} ( {t}_{i} + t ) ]}^{2} \qquad ( 1 )$$

This measurement is closely associated with the variance of the gradients of these channels.

With the aid of the lower curve, FIG. 5 shows the better distinction between QRS properties and other signal components such as the T wave when r(t) is compared with an ECG trace. The upper curve is the channel I, which is imaged for comparative purposes.

However, this new signal cannot be sufficiently regarded as a reliable marker, since it is possible that instead of one peak, two or more peaks can occur and then cause ambiguity in the determination of the reference instant. In order to become unique, it is necessary to determine a universal time, and this is achieved when the “temporal center of gravity” of this “derived” QRS property is calculated as follows:

$${T}_{ref} = {T}_{0} + \frac{{\sum }_{{t}_{i} = {T}_{0}}^{Te} r ( {t}_{i} ) {t}_{i}}{{\sum }_{{t}_{i} = {T}_{0}}^{Te} r ( {t}_{i} )} \qquad ( 2 )$$

In r(t), the QRS property has a markedly good signal-to-noise ratio. Only the strong amplitudes contribute substantially to the sums in equation (2), and therefore a realistic reference instant that is virtually uninfluenced by the selection of the size of the time window can be determined very stably.

Assembling a Characteristic Heartbeat

Once the reference instants have been determined, as described above, the remainder of the alignment procedure can be carried out in a simple way: the individual heartbeat signals of channels V₁ to V₆ of all the phases must be laid over one another appropriately.

In many cases, realistic clinical ECG data include substantial noise and would result in a distorted, irregular/jagged BSPM. In order to obtain realistically smoothed results, it is therefore proposed to form suitably averaged signals. It is normal in BSPM diagnoses to use only the properties that are averaged over individual heartbeats. It is described below how it is possible in the case of this method to form suitably averaged signals (termed “characteristic heartbeat signal” below):

FIG. 6 shows how an averaged signal, that is to say the characteristic heartbeat signal for an electrode position, is obtained for a channel and a phase. The reference instants for each heartbeat are determined as described above, and the individual beats are temporally aligned with this reference time and laid one over another. In addition, it is useful to adapt the baselines of the individual heartbeat signals vertically. FIG. 6a is produced in this way.

The variations from heartbeat to heartbeat are clearly recognizable by comparison with the individual ECG patterns. These variations from heartbeat to heartbeat are not particularly pronounced within the time interval from the beginning of the P wave up to the end of the T wave, and it is possible to assume an equilibrium between depolarization and repolarization. This fact is a vindication of the inventive system.

Furthermore, it may be seen from the variation of the preceding T wave and the following QRS that an appreciable variation of pulse rates occurs even with these few heartbeats (of the order of magnitude of 10) of one phase (10 seconds). It is even possible to deduce a T-wave or heart-rate alternans for this individual patient from the two-fold accumulation of the preceding T-wave signals.

Finally, the characteristic heartbeat signal (corresponding to FIG. 6b) of this electrode position can be extracted by determining the median of all the individual signal amplitudes in relation to each time step. It would likewise be possible to take the mean value but the median is more effective in ignoring heartbeat signals affected by artifacts or extra-systolic heartbeat signals, and should therefore be preferred. The derived characteristic heartbeat signal obtained is shown in FIG. 6b for one electrode position.

FIG. 7 is yielded when the characteristic heartbeat signals for all the electrode positions such as are displayed in FIG. 2b are laid one over another. The data situation of this processing stage cannot, however, be used yet in order to generate a pseudo BSPM. The resulting graphic display would be substantially distorted and, in some cases, misleading.

As in the case of the conventional BSPM, it is important to carry out a correction of the baseline (vertical raising) for all the heartbeat signals. This is frequently no easy task and—when it is wrongly carried out—misleading results arise in the graphic display and, for example, in the T-wave endpoint detection.

The question is reduced to finding the instant at which the ECG is electrically mute. In fact, when the ECG signals of high amplitude resolution are observed and signals of spatially differing regions are laid one over another, it is found that the ECG is never really “mute”. This is chiefly the case for high heartbeat rates, in the case of which the T wave and/or U wave merge with the P wave of the following heartbeat. The example in FIG. 7 is enough to show the problem, although it would be easy to demonstrate even more problematic cases.

FIGS. 8a and 8b show differing results for the setting of the baseline for the times Tₐ and T_{b}, characterized in FIG. 7. Upon a first glance, the instant T_{b} would be used as the most obvious choice, since it is situated furthest to the rear of the repolarization phase of the preceding heartbeat and just in advance of the atrial excitation. The entire excitation should be quietest electrically at this time.

If a spatial distribution of electric potential differences still exists at this instant, this “correction” of the baseline would cause a distortion of all the individual channel signals and could “create” image patterns, for example for the period between P wave and the beginning of the Q wave that do not really exist. A comparison of FIG. 8a with FIG. 8b shows just such an effect. The propagation of the signal amplitudes during this period between atrial and myocardial excitation is stronger in FIG. 8b than in FIG. 8a.

A further reason for caution can be derived from the additional signal in FIG. 7 (the single line over the majority of the signals). This signal is the variance of all the gradients of the signals situated underneath. In other words: were the rate of change in signal amplitude the same for all electrode positions, the variance would vanish. Consequently, a minimum in this additional signal specifies the time of minimal electrical activity.

Although the instant Tₐ is possibly not the ideal choice for the correction of the baseline, it does seem more apt than T_{b} and was selected to compile FIG. 9, the final “characteristic heartbeat”, which includes the synchronized signals of all the thorax leads.

Compilation of Pseudo BSPMs

As described below, a multiplicity of different BSPM plots could be generated from the characteristic heartbeat of FIG. 9. FIG. 10a shows the individual ECG signals for each electrode position, represented in a rectangular coordinate system, in agreement with FIG. 1b. The cursor marks the instant of |t|ₘₐₓ. As shown in FIG. 10b, a BSPM plot can be compiled in a contour line representation on the basis of these amplitude values.

Two different graphic displays for the same instant of the maximum T-wave amplitude in FIG. 9 are shown in FIG. 10b and FIG. 10c. The grid points are arranged in a square grid fashion in FIG. 10b. The numerical designation of the corresponding electrode positions, illustrated in FIG. 1b, are placed thereover. The potential field distribution is thus distorted intentionally in order to obtain a rectangular standard display that is easier to handle mathematically and/or graphically. In FIG. 10c, the coordinates of the grid points have been fixed geometrically in direct reference to FIG. 1b such that they display less distortion and a more realistic potential field distribution. Even if FIG. 1b is a type of 2D projection of a 3D reality, it still does contain appreciable distortion. In addition, significant artifacts, chiefly at the edges, are necessarily added by the triangulation and extrapolation processing. The display type in FIG. 10b can therefore be preferred.

FIG. 11 shows two sequences of graphic displays that visualize the development of the depolarization phase and repolarization phase, that is to say nine time steps of equal length shown by the cursor. An even more impressive image of the dynamics results from a combination of such displays (at a higher measuring rate) to form a video clip.

Finally, a so-called QRST integral display is also possible in which the amplitude values sₙ of the surface function at the grid points are the individual integrals

$${s}_{n} = {\int }_{{T}_{onset}}^{Tend} \vert {a}_{n} ( t ) \vert ⅆ t \qquad ( 3 )$$

of the individual characteristic heartbeat signals of the corresponding electrode positions n.

FIG. 12 shows the result of the spatial frequency distribution of the maxima or minima of 300 QRST integral plots.

It follows that most display types that are known from the traditional BSPM are possible in the case of the method presented here.

As already mentioned above, at least one validity characteristic can be determined in the case of one embodiment of the inventive system or the inventive method. This serves the purpose of checking whether the heartbeat pattern is constant during all eight measurement phases. To this end, the data processing system 30 is used to determine the variance of the R-R intervals and/or the QT times of all the heartbeats from the extremity leads sensed with the aid of the second measuring means 20. If this variance exceeds a specific threshold value (for example 5% of the associated mean value), the result of the examination should be rejected. Alternatively, the validity characteristic can also be ascertained by comparing the mean values with the R-R intervals and/or the QT times for one measurement phase with the associated mean value for all the measurement phases. If one of the mean values of the individual phases deviates by more than a specific threshold value (for example 5%) from the associated global mean value, the measurement should be rejected. It would be possible in principle to fashion a validity variable alone or else additionally by means of the thorax readings, in which case it is to be considered that a variation in the signal pattern could arise owing to the displacement of the electrodes.

The inventive method and system can be used to approximate the display of a conventional body surface potential mapping (BSPM) to a high degree. These results can be achieved with the aid of any commercial standard 12-channel ECG unit having digital data output. The compilation of a so-called characteristic heartbeat and graphic output of pseudo BSPM is possible with only a few calculations. Most digital 12-channel ECG units permit this method to be carried out simply by updating the software by a few algorithmic modules.

The pseudo BSPM can contribute clinically relevant information to the standard 12-channel ECG. It should be borne in mind that all the 12-channel ECG data are completely and automatically included in the recording of the pseudo BSPM, compare phase 1 in FIG. 3. The pseudo BSPM is commercially attractive since it causes no appreciable additional costs against the 12-channel ECG system. If the pseudo BSPM becomes popular, it should give rise to renewed interest in true BSPM and thereby assist in its spreading and its application.

### DESCRIPTION OF THE FIGURES

FIG. 1

Electrode positions a) for the breast electrodes of a conventional 12-channel ECG and b) for the inventive system. Positions 37 to 42 are adjacent to positions 30, 24, 6, 12, 18, 43 but laterally on the thorax.

FIG. 2

Schematic illustration of the thorax and extremity leads for the inventive system.

FIG. 3

ECG signals that are obtained during the first three phases of a recording session. The signals of the extremity electrodes (I, II, III, aVR, aVL, aVF) have been recorded continuously, while the breast electrodes are displaced upward between different phases, as illustrated in FIG. 1b. The individual signal traces are characterized by the corresponding electrode positions. The vertical cursor lines mark reference instants t₁, t₂, t₃.

FIG. 4

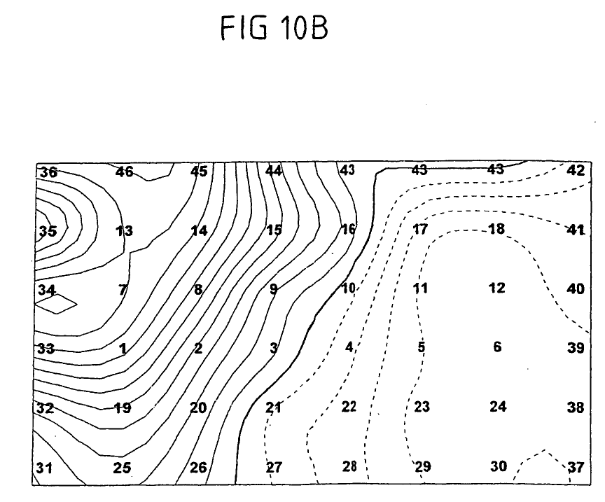

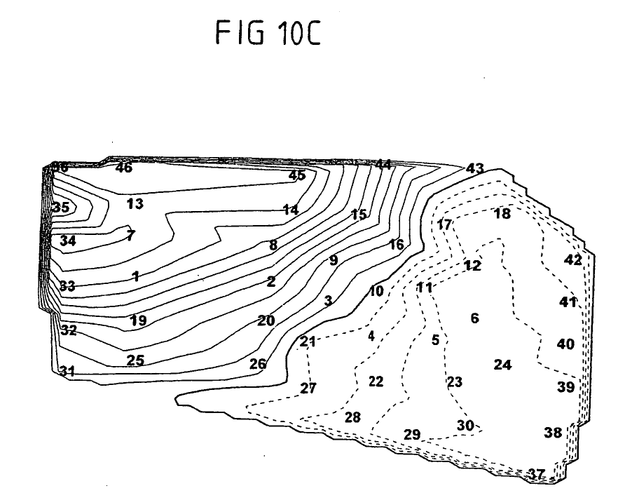

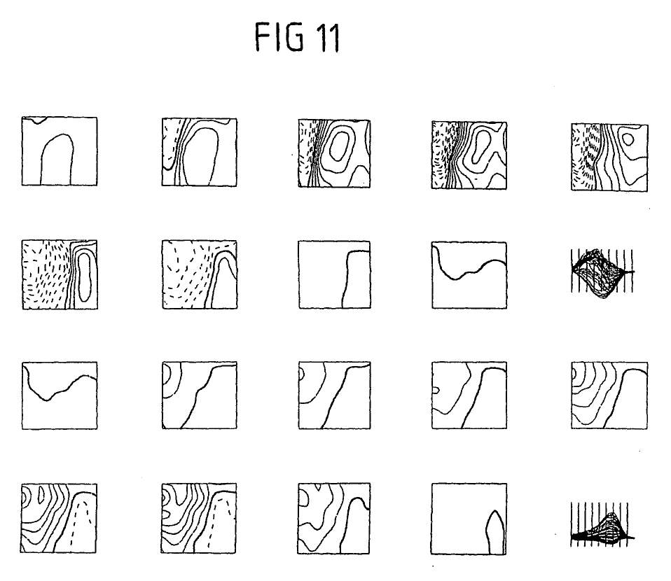

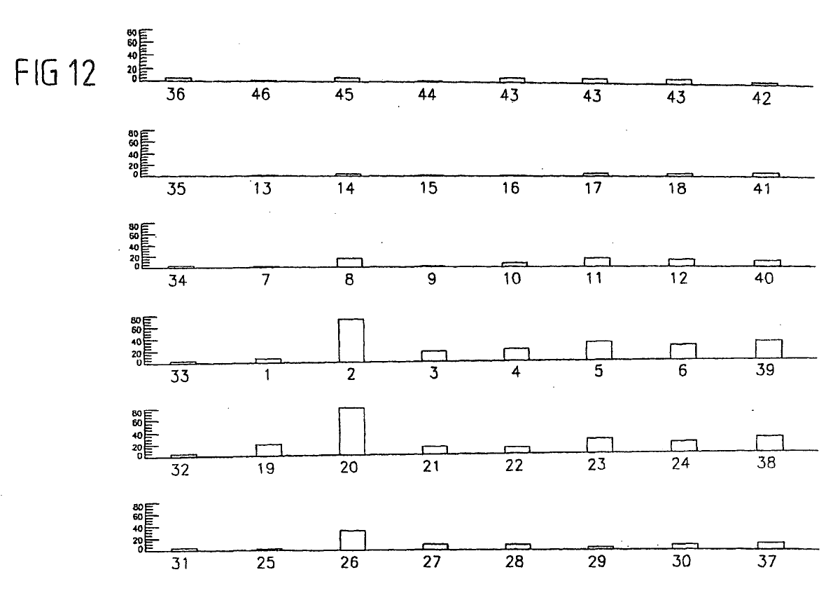

Display of the effect of various definitions of the reference instants on the resulting BSPM plots: a) real temporal alignment of the QRS signals of various electrode positions. The dashed and dotted cursor lines serve the purpose of alignment. b) associated BSPM plot. Here the thick continuous line represents the zero line. The continuous lines are positive potential value, the dashed lines are negative potential lines. c) temporal alignment of the same signals with the aid of the signal peak as adaptation criterion. d) resulting BSPM plot, the same convention applying to the equipotential lines as to FIG. 4b.

FIG. 5

Determining the reference instant T_{ref}. The signal of channel “I” is added at the top for the purpose of comparison (raised upward by 600 a.u.). The lower trace is the result of the application of equation (1) for all extremity signals. T_{ref} is fixed in the bounds T₀ and Tₑ by equation (2).

FIG. 6

Determining a characteristic individual heartbeat signal for one electrode position. a) aligning and laying one over another of all the heartbeat signals within a phase for one electrode position. b) median of the signal extracted from a).

FIG. 7

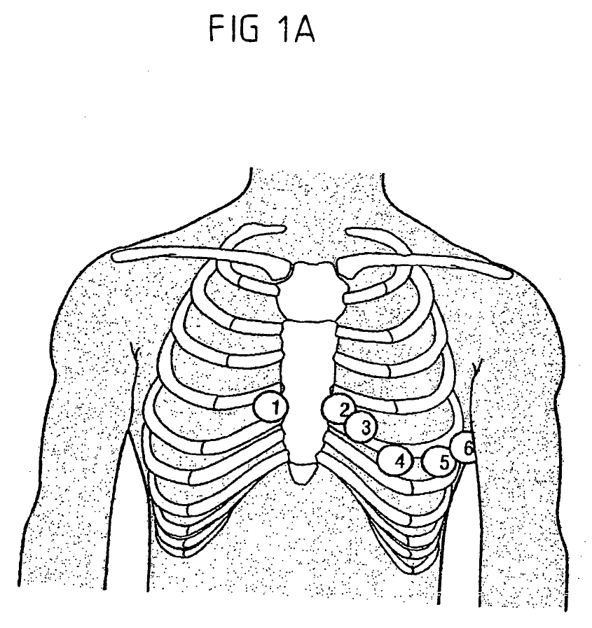

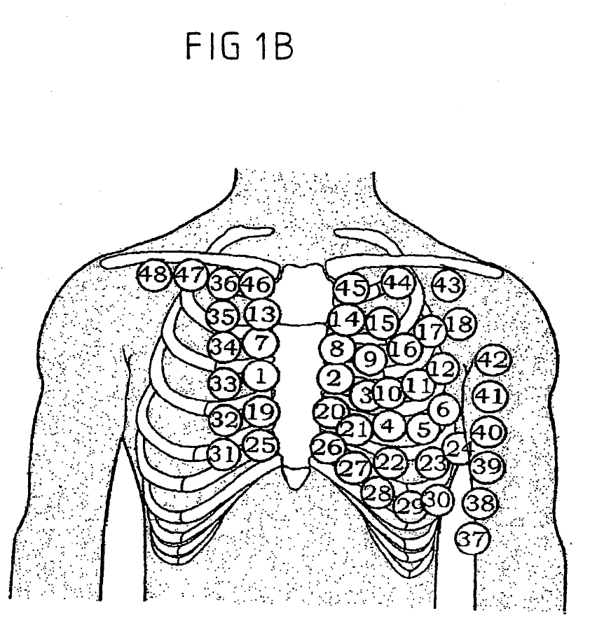

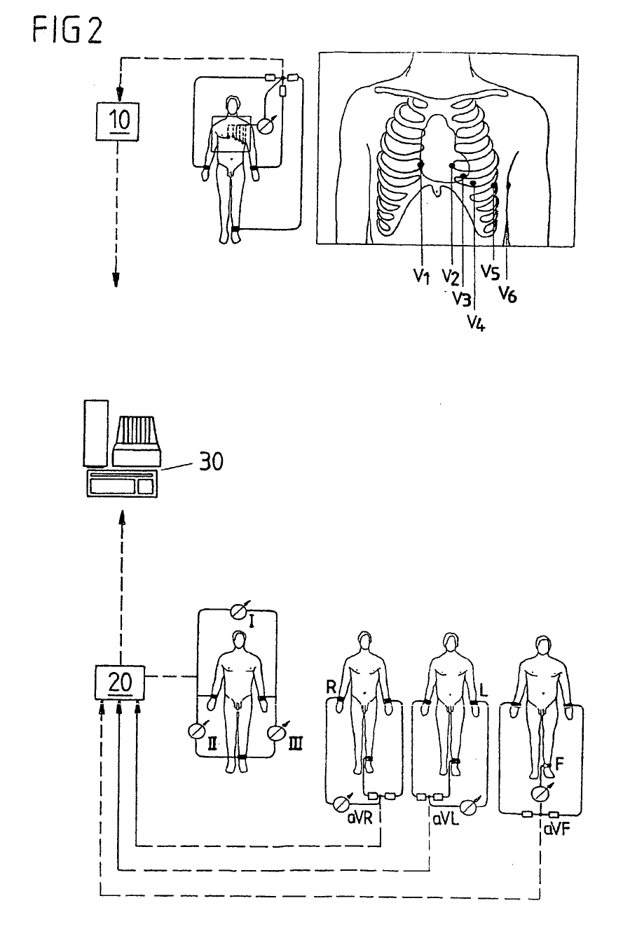

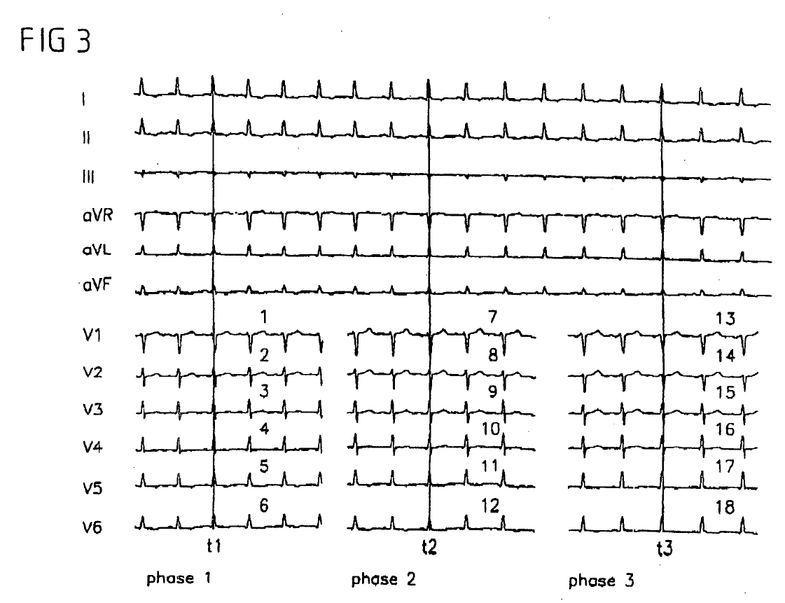

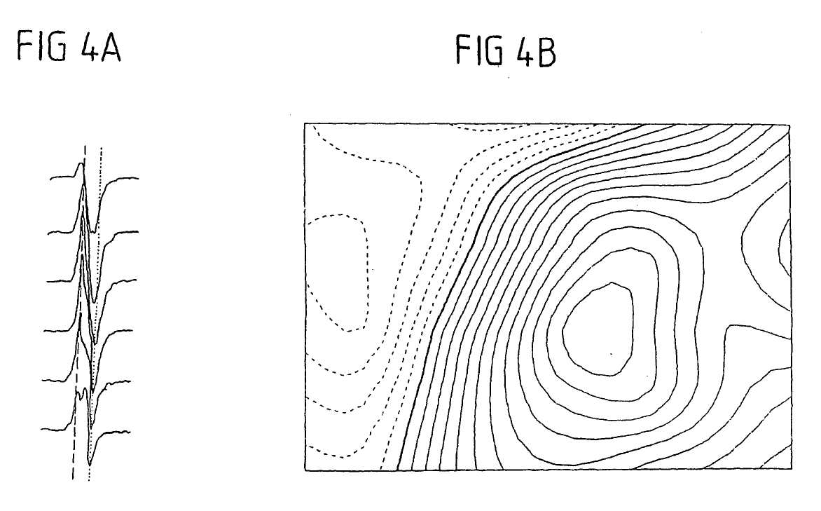

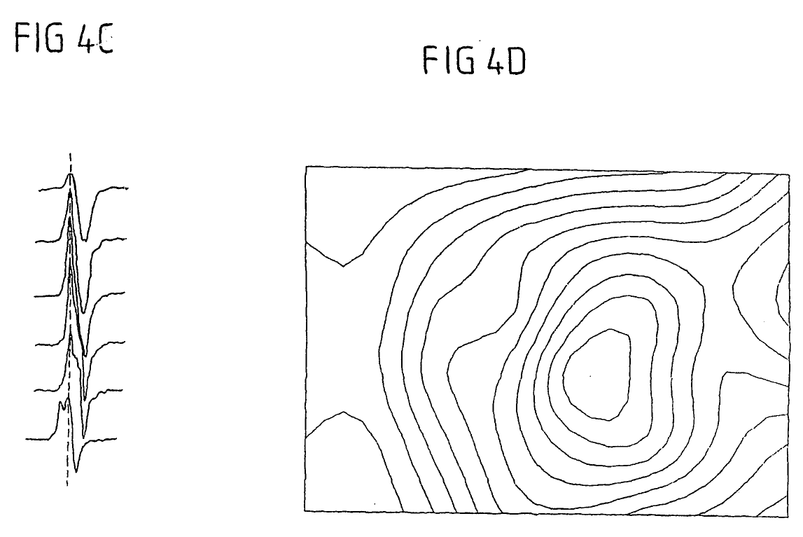

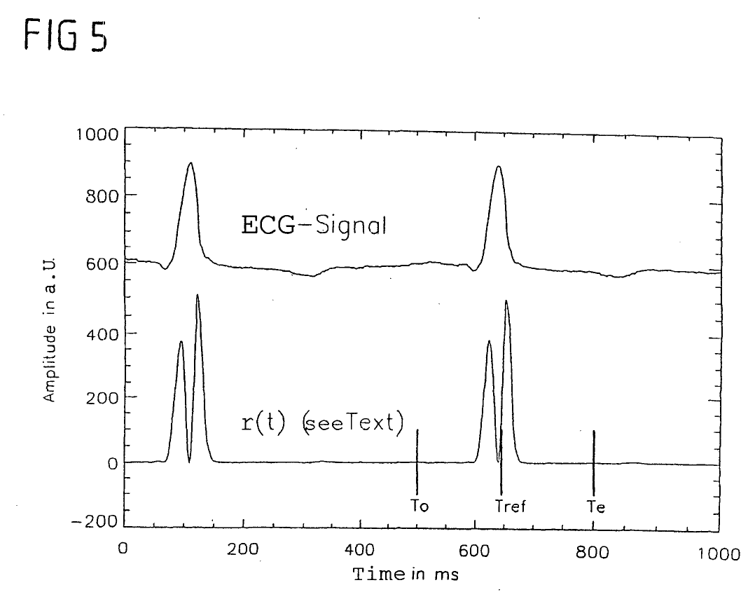

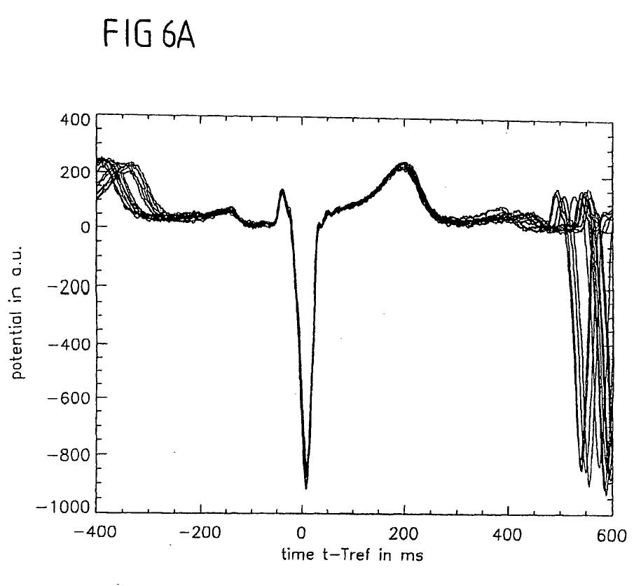

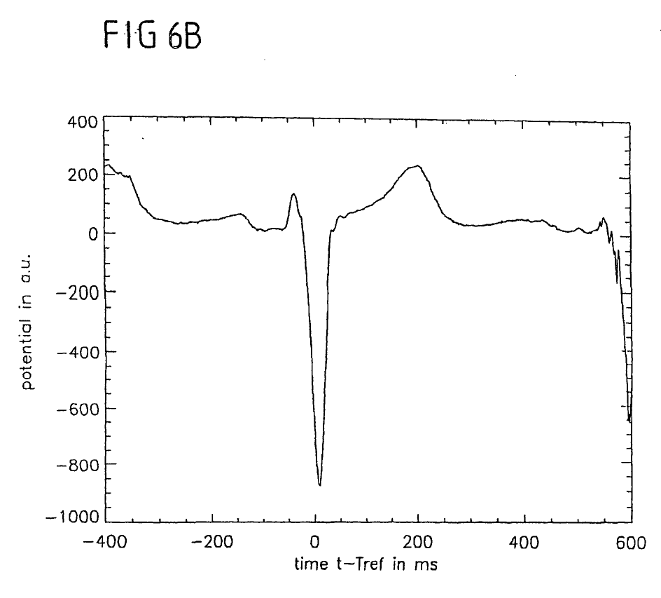

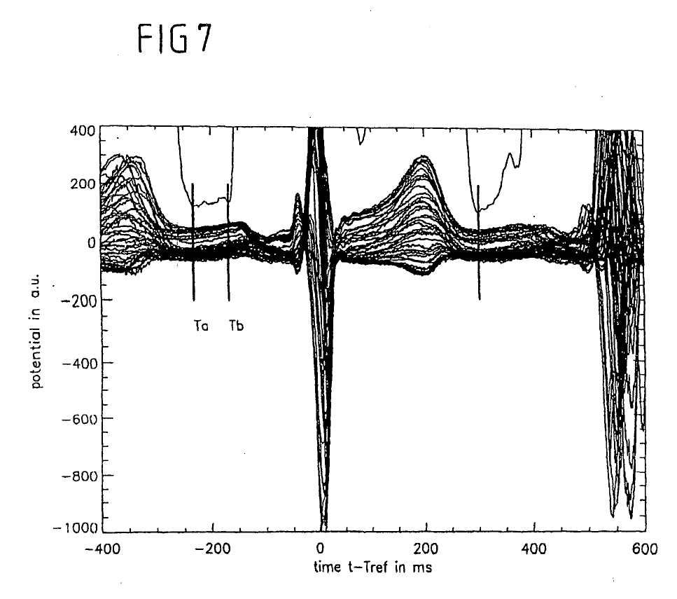

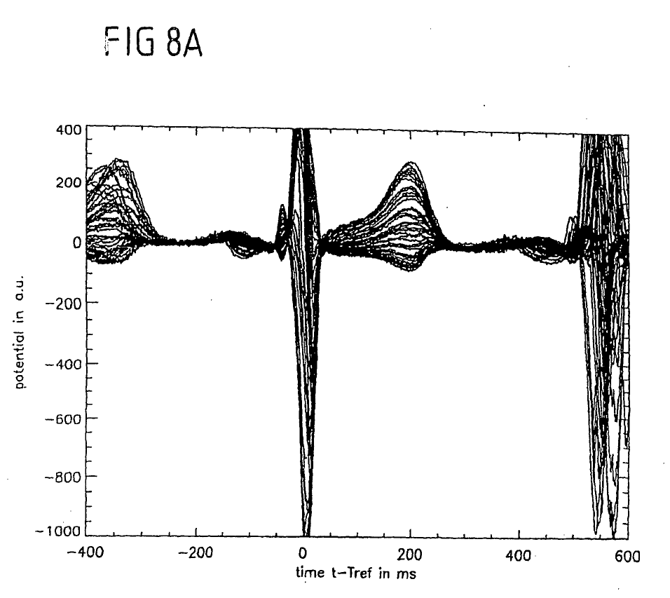

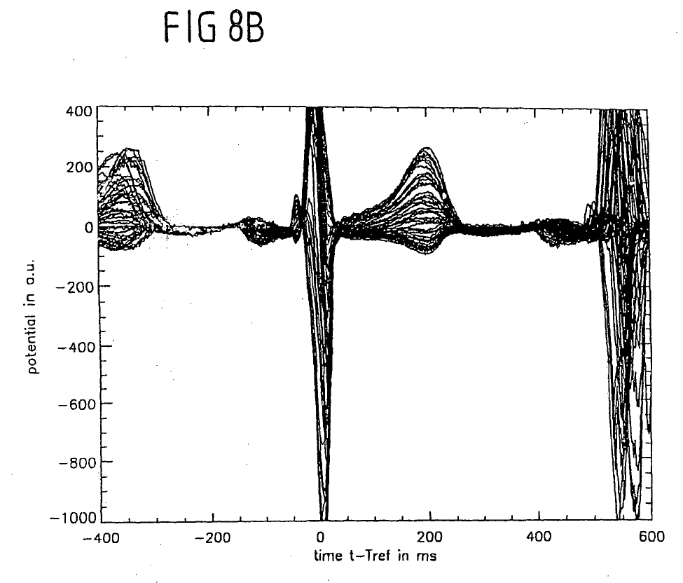

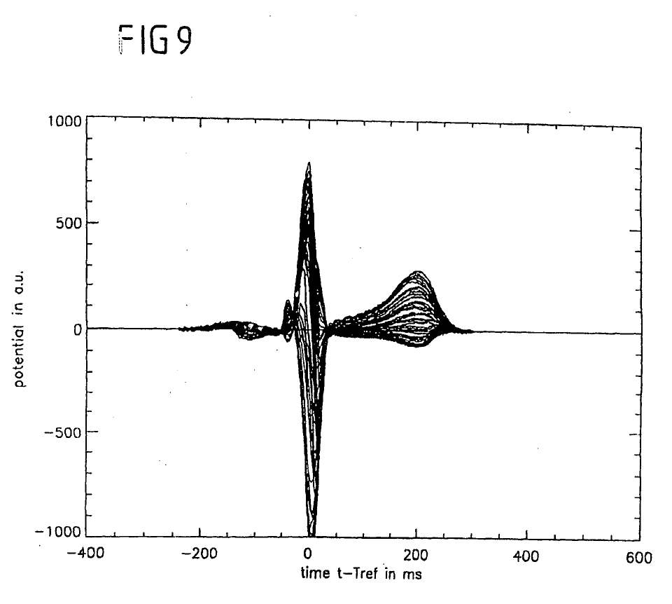

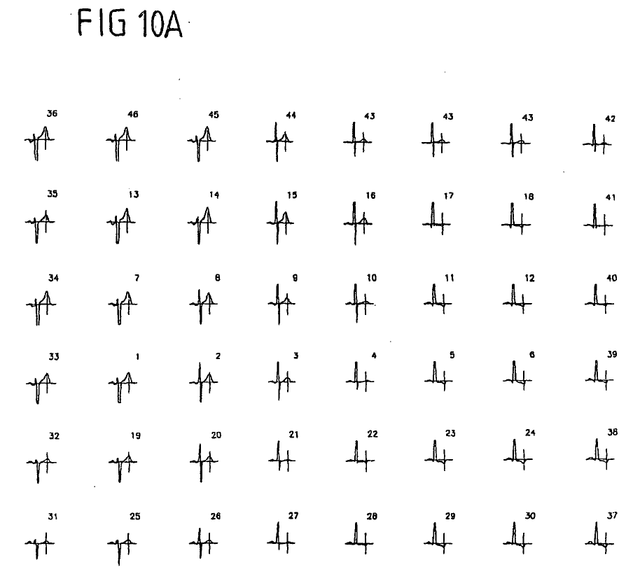

Superposition of the median signals of all 48 electrode positions of FIG. 1b). There is also an additional trace, which was obtained from the variance of all the gradients of the signals situated therebelow. The cursors mark the number of different instants that come into consideration for a baseline correction.

FIG. 8

Displays with corrected baseline: correction a) with reference to the instant Tₐ in FIG. 7, and b) with reference to T_{b}.

FIG. 9

Final display of a characteristic heartbeat.

FIG. 10

Producing a pseudo BSPM from the characteristic heartbeat in FIG. 9 at the instant of the T-wave maximum: a) ECG signals of all the channels arranged geometrically in accordance with the grid in FIG. 10b. The cursor marks the instant Tₘₐₓ. The signal amplitudes of this instant are represented as potential lines in FIG. 10b. In c), the grid is not arranged rectangularly but in accordance with FIG. 1b. The numerical designation of the respective electrode positions are marked at the grid points. The same convention as for FIG. 4b holds true for potential lines of FIGS. 10b and 10c.

FIG. 11

Display of the spatial temporal development of the repolarization phase of the characteristic heartbeat of FIG. 9 by means of a sequence of BSPM plots (represented with the aid of potential lines in accordance with the convention of FIG. 4b) for equidistant instants during the QRS complex (top) and the T wave (bottom) marked by the cursors in the respective ECGs.

FIG. 12

Example of the distribution of BSPM properties with reference to the electrode positions for 300 patients: for each electrode position (electrode numeral specified on the x axis), the height of the allotted bar is a measure of the number of the patients whose integral QRS image maxima or minima have fallen onto the corresponding electrode position. Only 28 percent of the maxima or minima correspond to the conventional breast electrodes V₁ to V₆.

1. An ECG system for large-surface recording of ECG signals, characterized by a first measuring means (10) for generating a first measured data record including at least one reading of the cardiac currents, at least one lead site of the first measuring means (10) being variable during the recording of the large-surface ECG signals, a second measuring means (20) for simultaneously generating a second measured data record including at least one reading of the cardiac currents, the lead site of the second measuring means (20) being spatially invariable during the recording of the large-surface ECG signals in order to obtain continuous measurement results, and a data processing system (30) having a means for synchronizing at least two signals, determined in a temporally offset fashion, of the first measured data record with at least one continuously detected signal of the second measured data record.

2. The ECG system as claimed in claim 1, characterized in that the first measured data record includes measurements of cardiac currents that have been obtained at thorax leads (V₁-V₆).

3. The ECG system as claimed in claim 1, characterized in that the first measured data record includes measurements of the cardiac currents from a temporal sequence of thorax leads (V₁-V₆) at different thorax positions.

4. The ECG system as claimed in claim 1, characterized in that the second measured data record includes at least one measurement of the cardiac currents of an extremity lead (I, II, III, aVR, aVL, aVF).

5. The ECG system as claimed in claim 4, characterized in that the second measured data record includes signals of the cardiac currents of all the extremity leads (I, II, III, aVR, aVL, aVF).

6. The ECG system as claimed in claim 1, characterized in that the synchronization is performed with the aid of at least one prominent signal pattern of the second measured data record.

7. The ECG system as claimed in claim 6, characterized in that the means for synchronizing uses the signal of an R wave in the second measured data record for the purpose of synchronization.

8. The ECG system as claimed in claim 7, characterized in that the means for synchronizing uses the signal of the rise in the R wave in the second measured data record for the purpose of synchronization.

9. The ECG system as claimed in claim 6, characterized in that the means for synchronizing uses prominent signal markers of a number of measured ECG channels.

10. The ECG system as claimed in claim 1, characterized by a filter, a means for averaging and/or for determining the median for signals of the first measured data record and/or of the second measured data record.

11. The ECG system as claimed in claim 1, characterized by a means for correcting the baseline of individual cardiac currents.

12. The ECG system as claimed in claim 1, characterized in that the data processing system (30) can use the amplitude values of all the thorax readings to determine a graphic display of the instantaneous potential distribution automatically for any desired instant of a measurement relative to a time reference obtained by means of a signal of the second measured data record.

13. The ECG system as claimed in claim 12, characterized in that the graphic display is a QRST integral map display.

14. The ECG system as claimed in claim 1, characterized in that the first measuring means (10) and/or the second measuring means (20) are/is arranged in a contrivance, in particular a vest, that can be worn on the human body.

15. The ECG system as claimed in claim 1, characterized in that a variance of measurement results can be ascertained as a validity characteristic by means of the data processing system (30).

16. The ECG system as claimed in claim 15, characterized in that the variance of the measurement results can be ascertained with the aid of a measure of specific ECG potential levels, in particular R-R intervals, QT times and/or of a comparison of a mean value of a measure of an ECG potential level of one measurement phase with the mean value for measures of ECG potential levels of all the measurement phases.

17. A method for large-surface recording of ECG signals, characterized by recording at least one first measurement of the cardiac currents with the aid of a first measuring means (10), at least one lead site of a first measuring means (10) being varied during recording of the large-surface ECG signals, simultaneously recording at least one second measurement of the cardiac currents with the aid of a second measuring means (20), the lead site of the second measuring means (20) being spatially invariable during recording of the large-surface ECG signals for the purpose of continuous measurement, the first and second measuring means (10, 20) generating a first measured data record and a second measured data record,
and immediately or at a later instant, at least two signals, determined in a temporally offset fashion, of the cardiac currents of the first measured data record being automatically synchronized in a data processing system (30) with at least one continuously determined signal of the second measured data record of the cardiac currents.

18. The method as claimed in claim 17, characterized in that at least two first readings are obtained on the thorax in a fashion separated by an intercostal spacing, in particular for the purpose of simulating a body surface potential mapping.
